(19) United States
(12) Patent Application Publication
(10) Pub. No.: US 2026/0137959 A1
(43) Pub. Date: May 21, 2026

(54) MULTICRITERIAL TREATMENT PLAN OPTIMIZATION USING LET COST FUNCTIONS

(71) Applicant: Elekta, Inc., Atlanta, GA (US)

(72) Inventors: Martin Soukup, Ebensee (AT); Kun-Yu Tsai, Shanghai (CN); Shoujian Xiong, Shanghai (CN); Raymond Philip Dalfsen, Adelaide, South Australia (AU)

(21) Appl. No.: 19/120,042

(22) PCT Filed: Oct. 21, 2022
(86) PCT No.: PCT/CN2022/126800

§ 371 Date: Apr. 10, 2025

(51) Int. Cl. A61N 5/10

(52) U.S. Cl. CPC A61N 5/1031; A61N 2005/1087

(57) ABSTRACT

System and techniques may be adapted for use in radiotherapy treatment planning. A technique may include determining a set of optimization functions with initial optimization goals, including at least one optimization function depending on LET and at least one optimization function for selecting a dose. The technique may include generating, for example using processing circuitry, a treatment plan via automated multicriteria optimization of the set of optimization functions while preserving the initial optimization goals using the patient information. In some examples, the treatment plan may be output (e.g., stored or displayed).

### BACKGROUND

Radiation therapy or “radiotherapy” may be used to treat cancers or other ailments in mammalian (e.g., human and animal) tissue. One such radiotherapy technique is referred to as “gamma knife,” by which a patient is irradiated using a number of lower-intensity gamma rays that converge with higher intensity and high precision at a targeted region (e.g., a tumor). In another example, radiotherapy is provided using a linear accelerator (“linac”), whereby a targeted region is irradiated by high-energy particles (e.g., electrons, high-energy photons, and the like). In another example, radiotherapy is provided using a heavy charged particle accelerator (e.g., protons, carbon ions, and the like). The placement and dose of the radiation beam is accurately controlled to provide a prescribed dose of radiation to the targeted region. The radiation beam is also generally controlled to reduce or minimize damage to surrounding healthy tissue, such as may be referred to as “organ(s) at risk” (OARs). Radiation may be referred to as “prescribed” because generally a physician orders a predefined dose of radiation to be delivered to a targeted region such as a tumor.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various examples discussed in the present document.

FIG. 1 illustrates an optimization block diagram, according to an example.

FIG. 2 illustrates a multicriterial optimization block diagram, according to an example.

FIG. 3 illustrates an example user interface for optimization, according to an example.

FIG. 4 illustrates a flowchart showing a technique for radiotherapy treatment planning, according to an example.

FIG. 5 illustrates generally an example of a system, such as may include a particle therapy system controller, according to an example.

FIG. 6 illustrates generally an example of a radiation therapy system, such as may include a particle treatment system and an imaging acquisition device, according to an example.

FIG. 7 illustrates generally a particle treatment system that may include a radiation therapy output configured to provide a proton therapy beam, according to an example.

FIG. 8 illustrates generally radiation dose depths in human tissue for various types of particles, according to an example.

FIG. 9 illustrates generally a spread-out Bragg Peak, according to an example.

FIG. 10 illustrates generally pencil beam scanning of an irregular shape volume from distal edge to proximal edge, according to an example.

FIG. 11 illustrates generally a diagram of an active scanning proton beam delivery system, according to an example.

### DETAILED DESCRIPTION

As discussed above, radiation therapy or “radiotherapy” is used to treat cancers or other ailments in mammalian (e.g., human and animal) tissue. Generally, ionizing radiation in the form of a collimated beam is directed from an external radiation source toward a patient. Modulation of a radiation beam may be provided by one or more attenuators or collimators (e.g., a multi-leaf collimator). The intensity and shape of the radiation beam may be adjusted by collimation avoid damaging healthy tissue (e.g., OARs) adjacent to the targeted tissue by conforming the projected beam to a profile of the targeted tissue.

In one approach, radiation therapy may be provided by using particles, such as protons, instead of electrons. This typically may be referred to as proton therapy. One significant known advantage of proton therapy is it provides superior dose distribution with minimal exit dose compared to other forms of radiation therapy, such as x-ray therapy. There is a significant reduction of dose to organs at risk (OAR) because of the minimal exit dose. Further advantages include lower dose per treatment, which lowers the risk of side effects and may improve quality of life during and after proton therapy treatment.

Higher LET values with comparable dose distribution may cause increased cell damage in proton radiotherapy as compared to lower LET values. Including LET in proton treatment planning optimization may improve cancer cell kill when increasing LET values in target structures and may cause less damage to healthy tissue when decreasing LET values in healthy tissue structures. However, no clinical protocols exist yet that specify the LET prescription. Currently, treatment planning goals rely primarily on dose distribution characterization.

With the addition of LET into optimization, the treatment planning problem includes an additional set of parameters. This increases the time a treatment planner needs to spend with tuning the treatment plan to achieve the desired LET behavior while keeping the existing dose goals.

The presently disclosed treatment planning includes automated multi-criteria optimization for dose objectives and constraints. The systems and techniques described herein solve the planning efficiency problem by including LET in the optimization process, and may include an automated multi-criteria optimization option. The improvement to the efficient of the workflow may include selecting LET objectives or constraints with relaxed initial prescription values and automatically improving the LET values by multi-criteria optimization while not harming selected dose or LET objectives or constraints. In some examples, instead of or in addition to directly using LET, one or more LET surrogates (e.g., DxLET based quantities) may be used.

An applied LET or LET surrogate specific cost function to achieve the objectives or constraints may be reused in some examples from an existing dose cost functions (e.g., dose volume histogram (DVH) point based cost functions, equivalent uniform dose (EUD) cost functions, etc.). In other examples, newly a designed cost function for LET purposes may be used (e.g., inverse serial cost function, etc.). Due to increased dose and LET distribution sensitivity of proton therapy to uncertainties (e.g., geometry, material and other uncertainties can lead to errors in dose and LET distribution), the LET or LET surrogate cost function may be robustly optimized simultaneously to already existing dose robustness optimization in some examples. Dose, LET or LET surrogate cost functions may be selected to be either multicriterial or not individually. To further improve treatment planning efficiency, any of the above choices or settings may be stored in a template, which may be applied to other patients.

In treatment planning, in some examples, a plan is created using dose-based optimization (e.g., using a cost function for the dose). Dose distribution is used as a primary measure that a radiotherapy physicist optimizes for during treatment planning. LET may be used as a further measure that the radiotherapy physicist may optimize. LET as a measure is less well established so the process of optimizing for LET may be limited to changes that do not affect the dose optimization. The present systems and techniques enable LET to be optimized without negatively impacting the optimized dose. This may be achieved by using multicriteria optimization (MCO).

Systems and techniques described herein may be used for automated treatment plan improvement by generating a treatment plan, including a set of optimization functions with initial optimization goals. In some examples, at least one optimization function may depend on linear energy transfer (LET), or at least one optimization function may be used for automated improvement of an optimization goal while preserving one or more initial optimization goals.

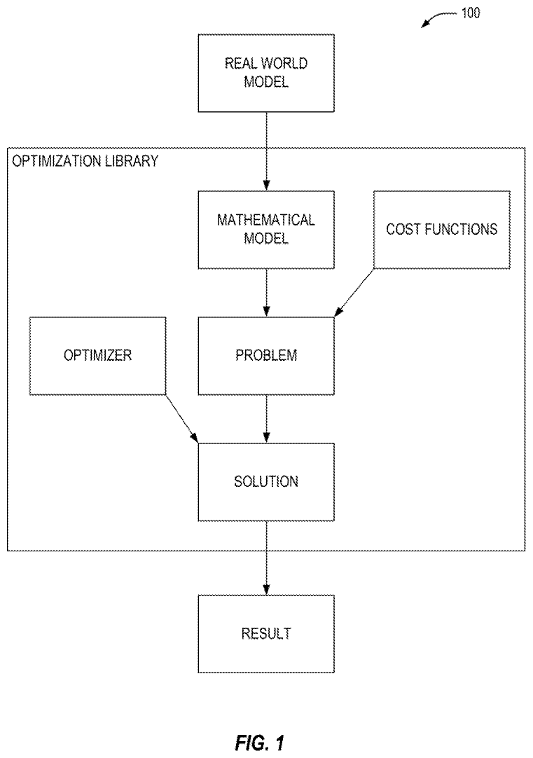

FIG. 1 illustrates an optimization block diagram 100, according to an example. The optimization block diagram 100 includes an optimization library, which receives a real world model input and outputs a result. The optimization library includes a mathematical model and one or more cost functions to define a problem. An optimizer is used to solve the problem, and a solution is output as a result.

When using optimization for solving real world problems, one challenge is to formulate the optimization problem, and another is how to interpret the solution. The real-world problem to be solved by this optimization library may be related to intensity modulated particle therapy (IMPT), where the goal of IMPT is to have more tumor control with less side effects. In other examples, the optimization may be related to proton arc therapy.

Given an optimization real world model, the optimization library of optimization block diagram 100 uses a problem component to transfer it to a mathematical model. The treatment goal may be described by one or more objective functions and constraints, for example depending on a dose deposited in the patient's body. The objectives and constraints are called cost functions. The cost functions and the mathematical model may be called the problem to be solved.

With the problem generated, now the optimization library may call an optimizer to obtain an optimal result. The optimization library may include multiple optimizers, for example a constrained-based conjugated gradient (CG) optimizer. Constrained-based means non target objectives are met before constraints are met. After the optimization is completed, a result with a map of nonnegative beamlet weights may be output.

Particles with a higher linear energy transfer (LET) are more likely to produce a biologically damaging effect to a given volume of tissue, due to a higher concentration of deposited energy. LET optimization allows a user to control the LET distribution in order to avoid high LET regions in an organ at risk, or while maximizing LET to a target, while maintaining a clinically acceptable dose distribution. A LET optimization function may be used with Proton Arc or Proton PBS delivery modes. In some examples, rather than using direct LET optimization, a product of dose and LET (LETxD) may be used in optimization.

In an example, a biological-like dose, b, may be used in a cost function, according to Eq. 1 below:

$$b = \sum c \times Li \times Di \qquad Eq . 1$$

In Eq. 1, Di and Li represent the dose and LET in individual voxels. The quantity b is computed for each cost function with the sum being performed over all the voxels relevant to the cost function being computed. The value of the constant c may be user specified. In some examples, c may be specific to a cost function. The quantity b may be interpreted as the biological extra dose that can be attributed to high LET. In some examples, c is both uncertain and dependent on tissue type and prescription dose level. Precise knowledge of the value of c is not necessary for use within the context of the optimization workflow where the parameter may be understood primarily as a way to alter the relative weight with which voxels at given dose and LET levels contribute to the overall cost function.

LET optimization cost functions may use dose optimization cost functions, in some examples. For example, any cost function available for dose optimization may be used, optionally excepting Target EUD or Target Penalty and Conformality. Dose optimization may be used to optimize the distribution of LET or LET xD. A LET cost function may be computed in a similar manner as a dose cost function, where in the LET or LET xD case, the input may be a 3D voxelized LET distribution rather than a dose distribution. To suppress noise in the LET distribution or to reduce the workload for the optimizer, a treatment planning system (TPS) may be used with a dose threshold setting for the LET cost function computation. Voxels whose deposited dose are below the threshold may be ignored in optimization.

In an example, a Target Serial cost function may be used (e.g., a cost function that is not a dose optimization cost function). The Target Serial cost function may be an inverse of a Serial cost function mathematically. This function may be defined according to Eq. 2 below:

$$f ( x ) = {( \frac{{X}_{0}}{x} )}^{N} \qquad Eq . 2$$

In Eq. 2, x represents LET or LETxD in a particular voxel, x0 represents the prescribed LET or LETxD, and N represents the power law exponent. This cost function may be used to increase the LET within the target region.

The optimization of dose and LET may be robust or non-robust. A robust optimization may include multiple dose distributions, while a non-robust optimization may include a single dose distribution. In non-robust optimization, each beam within a given plan may be considered to exist at a single, well specified point within a single patient model. Each beam thus is associated with a single dose distribution and there is a single total planned dose distribution, which is equal to the sum of the individual beam dose distributions. In the non-robust approach, the overall value of the cost function for a given plan may be fully determined from a single total dose distribution.

Robust optimization may improve on this functionality by accounting for one or more of various sources of dose differences, such as patient setup errors, density or material definition uncertainties, patient geometry changes, calculation parameters, or the like. The optimization framework may remove the assumption that each beam within a given plan exists at only a single point in space and within a single patient model. In the robust approach, each beam within a given plan may be associated with multiple dose distributions (and optionally LET or LETxD distributions if LET is requested). The cost function for the robust approach may be redefined in order to account for multiple possible total planned dose distributions or LET distributions.

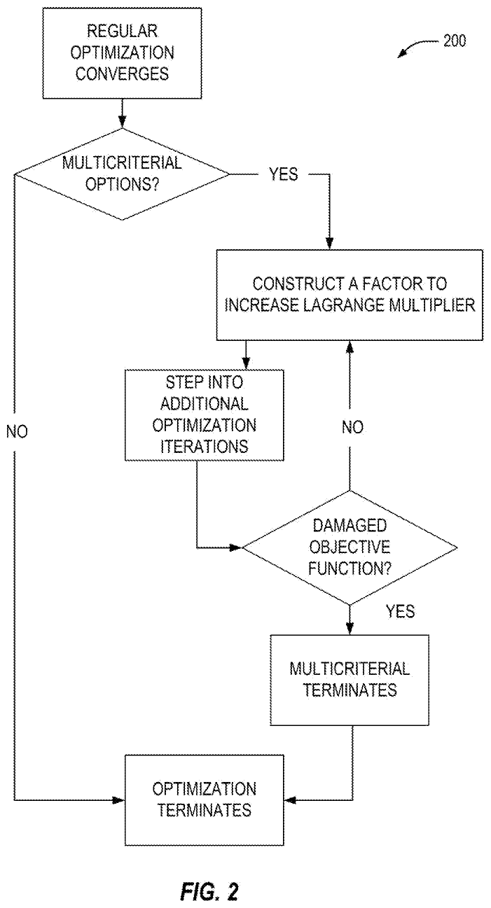

FIG. 2 illustrates a multicriterial optimization block diagram 200, according to an example. The multicriterial optimization block diagram 200 includes steps to achieve multicriterial optimization. After regular optimization converges, multicriteria options may be considered. When multicriteria options are not used, the optimization may terminate. When used, a factor to increase a Lagrange multiplier of a multicriterial on a cost function may be constructed, for example to lower an isoconstraint. After constructing a factor to increase the Lagrange multiplier, one or more additional optimization iterations may occur, subject to determining whether an objective function has been damaged. When damaged, the multicriterial optimization may terminate, ending optimization. When not damaged, the objective function may return to a previous step to construct a new factor to increase the Lagrange multiplier.

In a constrained mode, an optional parameter for an OAR or target constraint cost function is multicriterial. A treatment planning system (TPS) may treat this cost function as a secondary objective appended to the regular role of constraint. For example, after the regular constrained optimization, when the primary objectives are met well, the TPS may use a multicriterial approach to further minimize these multicriterial cost functions, while keeping each primary criterion satisfied. The process may continue until there are no further improvements available or until some specified time has occurred. Multicriterial optimization may be used when all objectives are achievable. Multicriterial optimization may be avoided when any higher priority criterion is not able to be met. When multiple multicriterial cost functions are selected, the TPS may construct a factor for each of these cost functions based on their conditions respectively, while increasing their weights in the additional optimization process.

In the constrained optimization mode, the optimizer attempts to raise dose in target structure to a desired coverage while keeping dose in an OAR below a defined level. The objective may obtain an optimal result while the constraints are strictly met. In some examples, when a loose constraint is set, the system may reduce dose to OAR as much as possible, while maintaining a prescribed dose to the target. One or more cost functions may be used to increase or decrease LET or LETxD. When optimizing, for a target cost function the aim is typically to increase the isoconstraint, while for an OAR cost function the aim is typically to decrease the isoconstraint.

In some examples, an available cost function may include Quadratic Overdose, Quadratic Underdose, Serial, Parallel, Conformality, Overdose DVH, Underdose DVH, Maximum Dose, Target Serial, or the like. For a Proton PBS plan or Proton Arc plan, optimization of the distribution of one or more of dose, LET, or LETxD may be used. The objectives of LET and LETxD optimization are similar, to avoid high values of LET in critical structures located within or near a target volume while limiting degradation of the best possible physical or biological dose distribution. When LET is selected, the system may optimize a dose-averaged LET. In order to reduce computational effort in optimization and remove regions of low importance, a dose threshold may be set to consider LET only in regions with dose higher than a dose threshold.

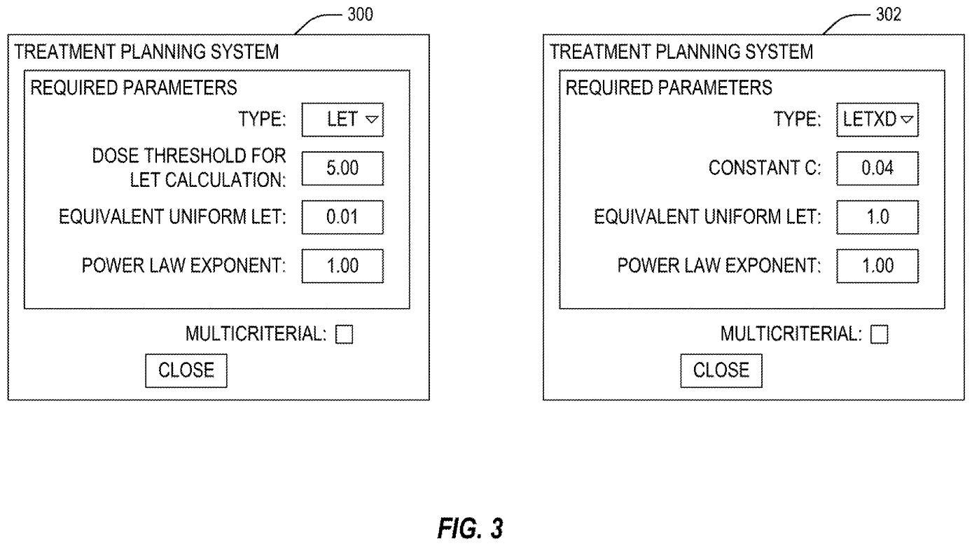

FIG. 3 illustrates an example user interface for optimization, according to an example. The example user interface may include a first state 300 when LET is selected for optimizing or a second state 302 when LETxD is selected for optimizing. The first state 300 includes an indication for entering a dose threshold for LET calculation. The second state 302 includes an indication for entering a constant c (e.g., as described above with respect to Eq. 1 in reference to FIG. 1). Both states 300 and 302 include indications for entering an equivalent uniform LET, entering a power law exponent, selecting multicriterial optimization, or closing the example user interface.

A Target Serial cost function may be used for LET and LETxD optimization. This cost function may not be usable for optimizing a dose distribution. The target serial cost function may be used to increase LET or LETxD in a structure volume to specific Equivalent Uniform LET or Equivalent Uniform LETxD value (according to the first state 300 or the second state 302, respectively).

When the type of Target Serial cost function is LET, as in the first state 300, the following parameters may be available:

| # | Parameter | Description |
|---|---|---|
| 1 | Dose Threshold | Cost function may be applied only to voxels of |
| | for LET | structure where dose is equal or higher than the |
| | Calculation(%): | defined dose threshold related to maximum dose |
| | | of plan, in some examples. |
| | | Range: 0.00 to 100.00. |
| 2 | Equivalent | Equivalent uniform LET value for the structure. |
| | Uniform LET | Equivalent Uniform LET follows the mathematical |
| | (keV/μm) | concept of Equivalent Uniform Dose. |
| | | Range: 0.01 to 100.0 (keV/μm) |

When the type of Target Serial cost function is LETxD, as in the second state 302, the following parameters may be available:

| # | Parameter | Description |
|---|---|---|
| 1 | c [μm/keV] | Enter the scaling factor to be used in the |
| | | formula (c * LET * Dose) |
| | | In case of LETxD, dose refers to physical dose. |
| | | Range: 0.00 to 100.00 |
| 2 | Equivalent Uniform | Equivalent uniform LETxD value allowed for |
| | Dose (cGy(RBE))/ | the structure. |
| | (Gy(RBE)) | Range: 0.0 to 20000.0 (cGy(RBE)) or |
| | | Range: 0.0 to 200.0 (Gy(RBE)). |

In either LET or LETxD (e.g., for either the first state 300 or the second state 302), the following parameters may be available:

| # | Parameter | Description |
|---|---|---|
| 1 | Power Law | This cost function may use one value that is the |
| | Exponent | Power Law Exponent (k) volume- effect |
| | | parameter. In general, when a small k value is |
| | | used, a large volume-effect is assumed. This |
| | | means that low LET/LETxD volumes and high |
| | | LET/LETxD volumes are approximately equally |
| | | weighted. When a large k value is used, there is |
| | | less tolerance for excessive damage to small |
| | | volumes of the assigned structure. In this case, |
| | | low LET/LETxD volumes receive a very small |
| | | weight relative to high LET/LETxD volumes. |
| | | When a particular k value is selected, a |
| | | complication model is selected based on the |
| | | desired outcome for the selected structure. |
| 2 | Multicriterial | With this option enabled, the system runs an |
| | | additional loop of optimization in an attempt to |
| | | increase delivered LET or LETxD while |
| | | maintaining target coverage. Fluence map |
| | | complexity may be increased as a result. |
| 3 | Shrink Structures | Structure Name: Shows the name of the |
| | | structure. Higher priority targets are shown |
| | | separately. |
| | | Include: Allows a shrink margin to be set for |
| | | the structure. Clicking the header allows for |
| | | selection or deselection of all or one or more of |
| | | the structures. To ignore the layering order and |
| | | optimize over all voxels, the check box is not |
| | | selected. |
| | | Link: Determine if wanted |
| | | Margin: Type the margin value applied the |
| | | structure. Individual structures may have |
| | | different margins if unlinked. |

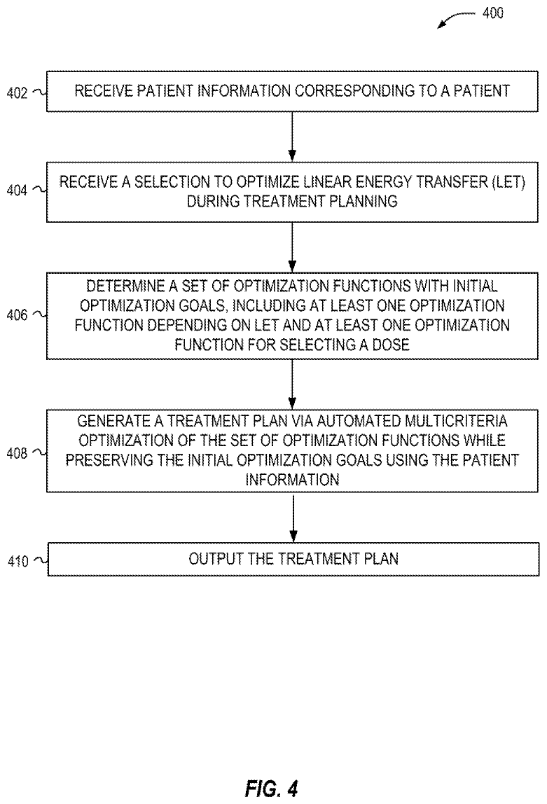

FIG. 4 illustrates a flowchart showing a technique 400 for radiotherapy treatment planning, according to an example. The technique 400 may be implemented using processing circuitry. The technique 400 includes an operation 402 to receive patient information corresponding to a patient. The technique 400 includes an operation 404 to receive a selection to optimize linear energy transfer (LET) during treatment planning.

The technique 400 includes an operation 406 to determine a set of optimization functions with initial optimization goals, including at least one optimization function depending on LET and at least one optimization function for selecting a dose, for example, in response to receiving the selection in operation 404. The at least one optimization function depending on LET may include using a LET surrogate. The at least one optimization function depending on LET may include a modified version of an existing dose cost functions. The initial optimization goals may include a goal to increase LET in a target or a target sub-region of the patient or a goal to decrease LET in an organ at risk or a sub-region of an organ at risk, in some examples.

The technique 400 includes an operation 408 to generate a treatment plan via automated multicriteria optimization of the set of optimization functions while preserving the initial optimization goals using the patient information. The treatment plan may include using proton arc therapy in some examples. In other examples, the treatment plan may include using intensity modulated proton therapy (IMPT). Operation 408 may include generating a robust optimization treatment plan, for example, where at least one beam of the robust optimization treatment plan is associated with multiple dose distributions. The technique 400 includes an operation 410 to output the treatment plan.

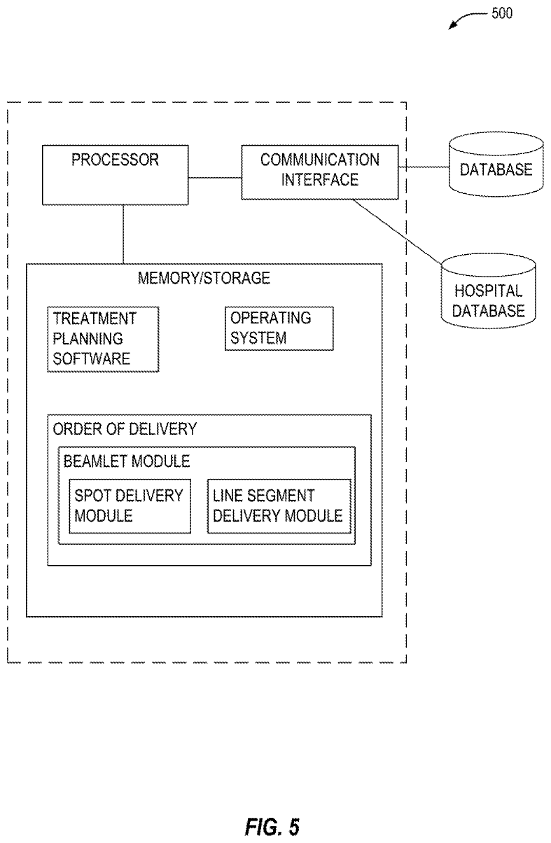

FIG. 5 illustrates generally an example of a system 500, such as may include a particle therapy system controller, according to an example. The system 500 may include a database or a hospital database. The particle therapy system controller may include a processor, communication interface, or memory. The memory may include treatment planning software, an operating system, or a delivery controller. The delivery controller may include a beamlet module for determining or planning spot delivery (e.g., using a spot delivery module) or line segment delivery (e.g., using a line segment delivery module).

In an example, the spot delivery module or the beamlet module may be configured to plan size of beamlets, location of a target or spot, or the like. The beamlet module may be used to determine an order of delivery of beamlets, for example in a spiral pattern as described herein. The order of delivery module may be in communication with the treatment planning software for planning delivery of beamlets. For example, the treatment planning software may be used to determine or plan gantry angle, gantry speed, beamlet size, spiral pattern (e.g., clockwise or counterclockwise), angle range for a particular spiral pattern (e.g., every ten degrees of the gantry rotation), or the like.

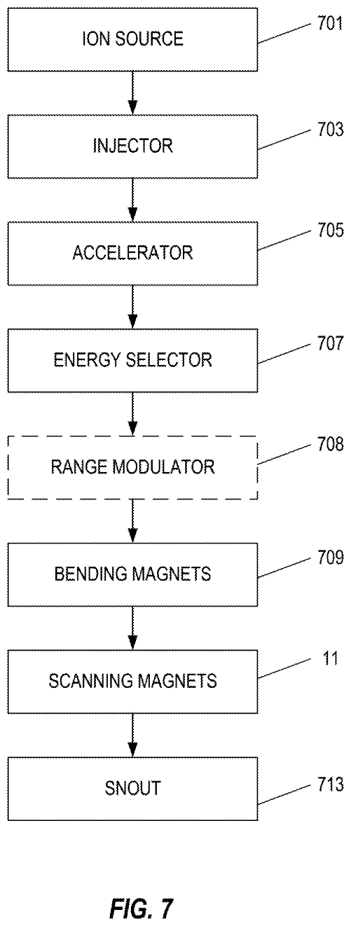

The processor may implement the plan, such as by communicating, via the communication interface or otherwise, to components used to implement the plan (e.g., to control devices or components, such as those described below with reference to FIG. 7). In an example, the communication interface may be used to retrieve stored information from a database or a hospital database (e.g., patient information, past procedure information for the patient or other patients, procedure instructions, information about particular devices or components, or the like).

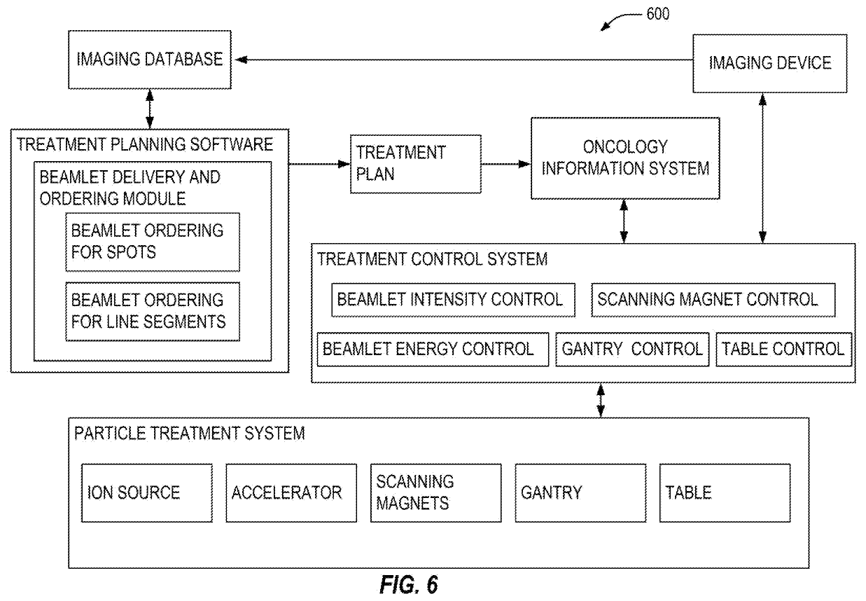

FIG. 6 illustrates generally an example of a radiation therapy system 600, such as may include a particle treatment system and an imaging acquisition device, according to an example. The particle treatment system includes an ion source, an accelerator, and scanning magnets, each of which is described in more detail below with respect to FIG. 7. The particle treatment system includes a gantry and a table, where the gantry may be mounted on the table, affixed to the table, or stabilized with respect to the table. The table may hold a patient. The gantry may be a rotating gantry, and may rotate with respect to the table (e.g., around the table) or with respect to the patient (and the table or a portion of the table may rotate with the gantry).

The particle treatment system may communicate with a treatment control system, which may be used to control actions of the particle treatment system. The treatment control system may communicate with an imaging acquisition device (e.g., to receive images taken by the imaging acquisition device or an imaging database) or an oncology information system. The oncology information system may provide treatment plan details to the treatment control system, such as received from treatment planning system. The treatment control system may use the treatment plan to control the particle treatment system (e.g., activate the gantry, the ion source, the accelerator, the scanning magnets, a particle beam, or the like). The treatment control system, for example, may include a beamlet intensity control, a beamlet energy control, a scanning magnet control, a table control, a gantry control, etc. In an example, the beamlet intensity control and the beamlet energy control may be used to activate a beamlet of a particular size or to target a particular location. The scanning magnetic control may be used to deliver beamlets according to the treatment plan, for example in a spiral pattern. The gantry control or the table control may be used to rotate the gantry.

The treatment planning software may include components such as a beamlet delivery and ordering module, with, for example, separate controls for beamlet ordering for spots or line segments. The treatment planning software is described in more detail above with respect to FIG. 5. The treatment planning software may access an imaging database to retrieve images or store information. When a treatment plan is completed, the treatment planning software may send the plan to an oncology information system for communication with the treatment control system.

FIG. 7 illustrates an example of a particle treatment system 700 that may include a radiation therapy output configured to provide a proton therapy beam. The particle treatment system 700 includes an ion source 701, an injector 703, an accelerator 705, an energy selector 707, a plurality of bending magnets 709, a plurality of scanning magnets 711, and a snout 713.

The ion source 701, such as a synchrotron (not shown) may be configured to provide a stream of particles, such as protons. The stream of particles is transported to an injector 703 that provides the charged particles with an initial acceleration using a Coulomb force. The particles are further accelerated by the accelerator 705 to about 10% of the speed of light. The acceleration provides energy to the particles, which determines the depth within tissue the particles may travel. The energy selector 707 (e.g., a range scatter) may be used to select the energies of the protons to be delivered to the patient. In an example called passive scattering, an optional range modulator 708 (e.g., also called a ridge filter or a range modulation wheel) may be utilized to broaden the beam to fit the tumor. After selecting energies, a set of bending magnets 709 may be utilized to transport the stream of protons into a radiation therapy treatment room of a hospital. Further, scanning magnets 711 (e.g., x-y magnets) are used to spread the proton beam to, or trace, an exact image of the tumor shape. A snout 713 is used to further shape the proton beam. In some examples, the stream of particles may be composed of carbon ions, pions, or positively charged ions.

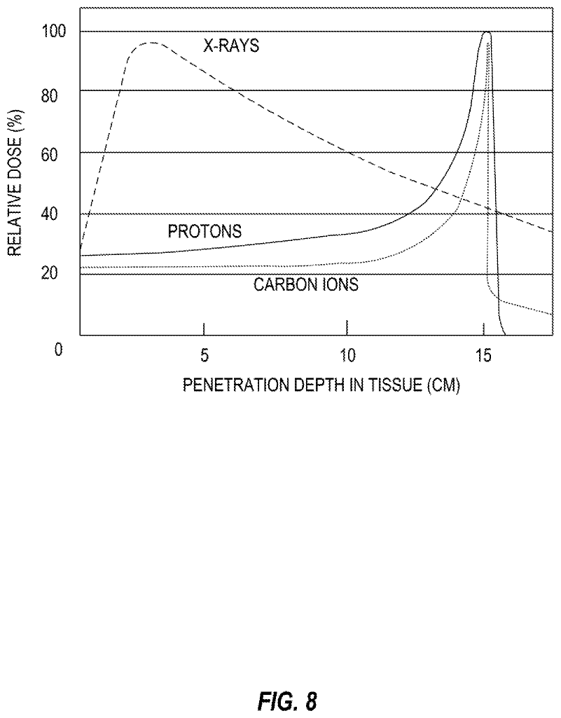

FIG. 8 provides an illustration of a comparison of radiation dose depths for various types of particles in human tissue. As shown, the relative depth of penetration into human tissue of photons (e.g., x-rays) versus protons versus carbon ions is provided (e.g., including any radiation dose provided at a distance beneath the surface, including secondary radiation or scatter). Each radiation dose is shown relative to the peak dose for a proton beam having a single energy which has been set to 100%.

The mono-energetic (e.g., single energy) proton beam indicates a plateau region starting at approximately 25% that gradually increases until approximately 10 cm depth in tissue where it rapidly increases to the Bragg Peak at 15 cm and then advantageously falls to zero within a short distance. No additional dose is delivered at the end of the Bragg peak.

The photon beam (e.g., labelled as X-rays) indicates the initial build up due to electron scatter (e.g., the primary means by which X-rays deliver dose to tissue is through transfer of energy to electrons in the tissue). This is followed by an exponential fall off, which continues past the distal edge of the target, which is at approximately 15 cm depth in the diagram. The x-ray beam has an entrance (skin) dose set to match that of the proton beam. With normalization (e.g., scaling) at 15 cm depth, the dose due to x-rays is at 40% of the dose provided by proton beam, while the x-ray beam has a peak dose of greater than 95% (“near” 100%) at approximately 3 cm depth. If the x-ray data is renormalized to achieve 100% dose at 15 cm, the peak dose at approximately 3 cm depth would be approximately 240%, in a location where dose is not desired (e.g., prior to the target). Therefore, with x-rays, a considerable amount of dose is delivered prior to the target and an appreciable amount of dose is delivered past the target.

The mono-energetic carbon beam shows a plateau region at the entrance dose that is lower than the proton beam. The carbon beam has a sharper Bragg Peak that falls more precipitously than the proton beam, but the carbon beam has a tail (e.g., known as a “spallation tail”, where some of the Carbon nuclei shatter into Helium ions) that has approximately 10% additional dose, or less, past the desired target by several centimeters. The carbon ion beam has an undesired entrance and skin dose compared to the proton beam, but the carbon ion beam has a non-trivial dose delivered past the target.

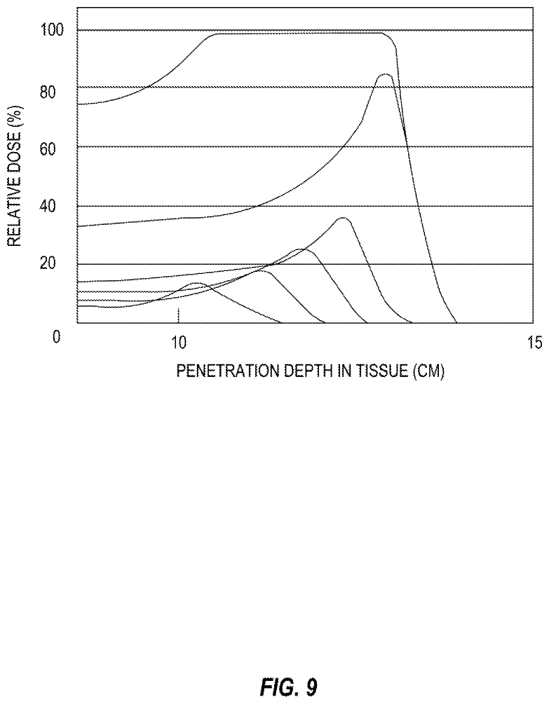

FIG. 9 provides an illustration of a spread-out Bragg peak (SOBP). The SOBP. displays a relative depth dose curve for the combination of a set of proton beams of various initial energies each of which has had some spread in energy (e.g., variable absorption of energy in tissue). The desired result of having a uniform dose for a target of a particular thickness. As shown, the target is shown with a proximal depth of approximately 10 cm, a distal depth of approximately 13 cm, and a target thickness of approximately 3 cm. Within the target, the dose is quite uniform (with an average normalized at 100%). The diagram does not start at 0 cm depth and is not explicitly showing the entrance (skin) dose, but the nature of the entrance region of proton beams is a relatively flat depth dose curve. Typically, the entrance (skin) dose will be approximately 70% of the target dose (e.g., shown at the far right edge of the x-axis). A SOBP may be obtained using a variety of approaches, including using a scattered proton beam with modulation of the energy (variable absorption) utilizing a variety of devices (e.g., a static ridge filter or a dynamic range modulation wheel), or by selection of a number of mono-energetic proton beams that do not undergo scatter.

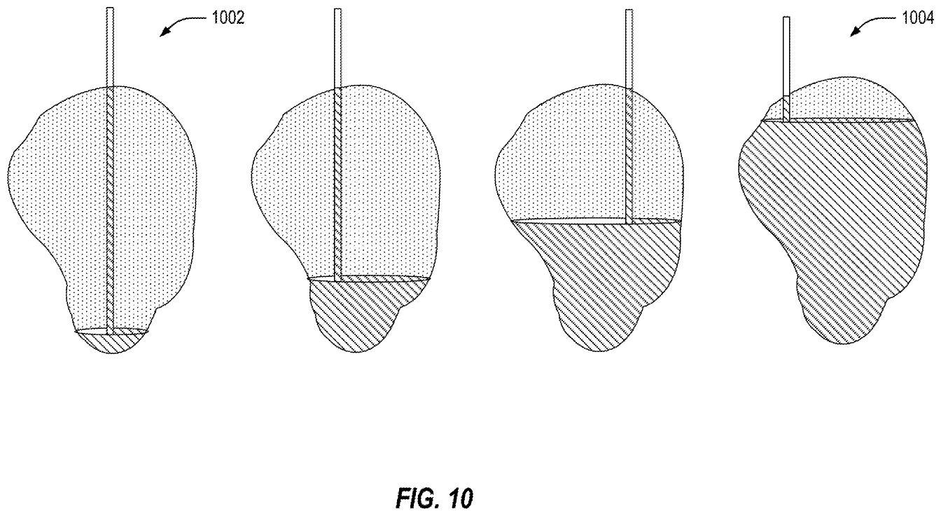

FIG. 10 provides an illustration of a Pencil Beam Scanning of an irregular shape volume from a distal edge (e.g., bottom) to a proximal (e.g., top) edge. As shown, the irregular shaped tumor volume is irradiated layers of protons. For example, a first time snapshot 1002 shows a first layer of protons being delivered, and a later time snapshot 1004 shows that most of the layers have been delivered. Each layer has its own cross-sectional area to which the protons having the same energy are delivered. The total radiation dose is provided as a layer-by-layer set of beamlets. Each layer of may have different energies. The most common means of specifying and delivering the set of beamlets to the cross-sectional area is to define and deliver beamlets having a constant diameter (“spot size”) to a selection of grid points on each layer. While the majority of the dose from the beamlet is delivered to the targeted layer, a significant amount of dose is delivered along the path to the targeted layer. The dose to proximal layers from beamlets defined for distal layers is accounted for in the specification of the beamlets defined for the proximal layers. The ability to individually specify the number of particles (e.g., the meterset) for a given beamlet ensures that each part of the volume being irradiate receives the desired dose.

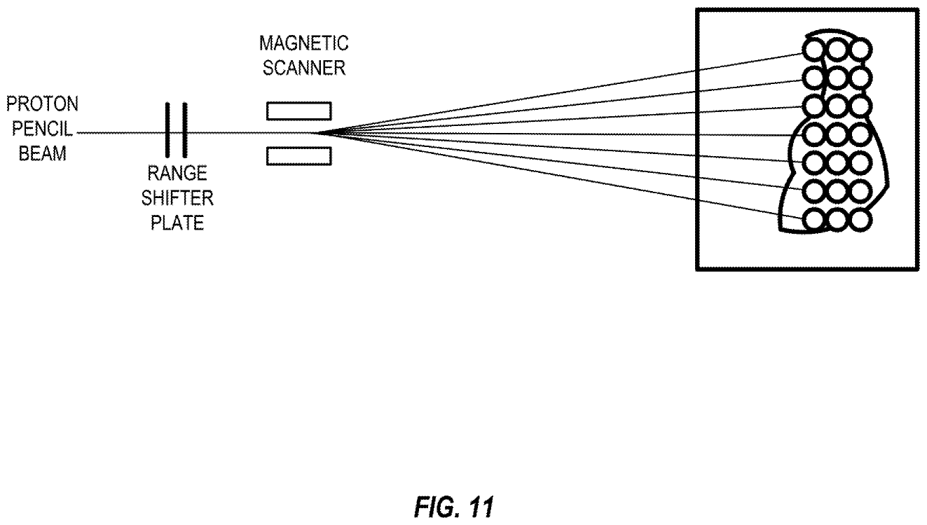

FIG. 11 provides an illustration of a diagrammatic representation of a typical active scanning proton beam delivery system. As shown, a single layer of a pencil beam scan is being delivered, with a grid of spots depicted on a patient in conjunction with a contour of the cross-sectional area to which particles are to be delivered. An incoming mono-energetic proton beamlet has a specified amount of its energy absorbed by the Range Shifter (e.g., in FIG. 11 it is a Range Shifter plate), resulting in a beamlet with the desired energy to achieve a certain depth for the Bragg Peak in the patient to treat the specified layer. A magnetic scanner, which has the ability to deflect the particles in both a vertical and a horizontal direction. The strength of the magnetic fields may be adjusted to control the deflection in the direction perpendicular to the magnetic field and the incoming beamlet. The rate at which the magnetic field strengths may be adjusted determines the rate at which the scanning may take place. For example, the intensity of the proton beamlet in combination with the scanning rate determines how much dose may be delivered to a specific area (e.g., in FIG. 11, a “spot”) in a particular amount of time (e.g., particles/unit area). In theory, the magnetic field strengths may be adjusted independently of each other (in a fashion similar to the children's toy “Etch a Sketch®”, provided by Spin Master™, Toronto, Canada; with the pencil beamlet intensity being a variable not available in the children's toy). The most common scheme for scanning is to scan in one direction quickly and to scan in the perpendicular direction more slowly in a raster fashion, similar to how early televisions were controlled (e.g., Cathode Ray Tube (CRT), which use electrons instead of protons), but arbitrary patterns may be scanned (similar to the previously mentioned toy). Delivery of distinct spots is achieved by incrementing the scanning magnetic field strength and throttling the pencil beam intensity between increments.

The above detailed description includes references to the accompanying drawings, which form a part of the detailed description. The drawings show, by way of illustration, specific examples in which the invention may be practiced. Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other examples may be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed example. Thus, the following claims are hereby incorporated into the Detailed Description as examples, with each claim standing on its own as a separate example, and it is contemplated that such examples may be combined with each other in various combinations or permutations. The scope of the invention should be determined with reference to the appended claims, along with the full scope of equivalents to which such claims are entitled.

Example 1 is a method for radiotherapy treatment planning, the method comprising: receiving patient information corresponding to a patient; determining a set of optimization functions with initial optimization goals, including at least one optimization function depending on linear energy transfer (LET) and at least one optimization function for selecting a dose; generating, using processing circuitry, a treatment plan via automated multicriteria optimization of the set of optimization functions while preserving the initial optimization goals using the patient information; and outputting the treatment plan.

In Example 2, the subject matter of Example 1 includes, wherein the treatment plan includes using proton arc therapy.

In Example 3, the subject matter of Examples 1-2 includes, wherein the treatment plan includes using intensity modulated proton therapy (IMPT).

In Example 4, the subject matter of Examples 1-3 includes, wherein generating the treatment plan includes generating a robust optimization treatment plan, and wherein at least one beam of the robust optimization treatment plan is associated with multiple dose distributions, multiple LET distributions, or multiple LET surrogate distributions.

In Example 5, the subject matter of Examples 1-4 includes, wherein the at least one optimization function depending on LET includes using a LET surrogate.

In Example 6, the subject matter of Examples 1-5 includes, wherein the at least one optimization function depending on LET is a modified version of an existing dose cost functions.

In Example 7, the subject matter of Examples 1-6 includes, wherein the initial optimization goals include a goal to increase LET in a target or a target sub-region of the patient.

In Example 8, the subject matter of Examples 1-7 includes, wherein the initial optimization goals include a goal to decrease LET in an organ at risk or a sub-region of an organ at risk.

Example 9 is at least one machine-readable medium including instructions for radiotherapy treatment planning, which when executed by processing circuitry, cause the processing circuitry to perform operations to: receive patient information corresponding to a patient; determine a set of optimization functions with initial optimization goals, including at least one optimization function depending on linear energy transfer (LET) and at least one optimization function for selecting a dose; generate a treatment plan via automated multicriteria optimization of the set of optimization functions while preserving the initial optimization goals using the patient information; and output the treatment plan.

In Example 10, the subject matter of Example 9 includes, wherein the treatment plan includes using proton arc therapy.

In Example 11, the subject matter of Examples 9-10 includes, wherein the treatment plan includes using intensity modulated proton therapy (IMPT).

In Example 12, the subject matter of Examples 9-11 includes, wherein to generate the treatment plan, the operations cause the processing circuitry to generate a robust optimization treatment plan, and wherein at least one beam of the robust optimization treatment plan is associated with multiple dose distributions, multiple LET distributions, or multiple LET surrogate distributions.

In Example 13, the subject matter of Examples 9-12 includes, wherein the at least one optimization function depending on LET includes using a LET surrogate.

In Example 14, the subject matter of Examples 9-13 includes, wherein the at least one optimization function depending on LET is a modified version of an existing dose cost functions.

In Example 15, the subject matter of Examples 9-14 includes, wherein the initial optimization goals include a goal to increase LET in a target or a target sub-region of the patient.

In Example 16, the subject matter of Examples 9-15 includes, wherein the initial optimization goals include a goal to decrease LET in an organ at risk or a sub-region of an organ at risk.

Example 17 is a system for radiotherapy treatment planning, the system comprising: processing circuitry; memory, including instructions, which when executed by the processing circuitry, cause the processing circuitry to: receive patient information corresponding to a patient; determine a set of optimization functions with initial optimization goals, including at least one optimization function depending on linear energy transfer (LET) and at least one optimization function for selecting a dose; generate a treatment plan via automated multicriteria optimization of the set of optimization functions while preserving the initial optimization goals using the patient information; and output the treatment plan.

In Example 18, the subject matter of Example 17 includes, wherein the treatment plan includes using proton arc therapy.

In Example 19, the subject matter of Examples 17-18 includes, wherein the treatment plan includes using intensity modulated proton therapy (IMPT).

In Example 20, the subject matter of Examples 17-19 includes, wherein the processing circuitry is further caused to receive a selection to optimize LET during treatment planning, and wherein to determine the set of optimization functions occurs in response to receiving the selection.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

1.-20. (canceled)

21. A method for radiotherapy treatment planning, the method comprising:
receiving patient information corresponding to a patient;
determining a set of optimization functions with initial optimization goals, including at least one optimization function depending on linear energy transfer (LET) and at least one optimization function for selecting a dose;
generating, using processing circuitry, a treatment plan via automated multicriteria optimization of the set of optimization functions while preserving the initial optimization goals using the patient information; and
outputting the treatment plan.

22. The method of claim 21, wherein the treatment plan includes using proton arc therapy.

23. The method of claim 21, wherein the treatment plan includes using intensity modulated proton therapy (IMPT).

24. The method of claim 21, wherein generating the treatment plan includes generating a robust optimization treatment plan, and wherein at least one beam of the robust optimization treatment plan is associated with multiple dose distributions, multiple LET distributions, or multiple LET surrogate distributions.

25. The method of claim 21, wherein the at least one optimization function depending on LET includes using a LET surrogate.

26. The method of claim 21, wherein the at least one optimization function depending on LET is a modified version of an existing dose cost functions.

27. The method of claim 21, wherein the initial optimization goals include a goal to increase LET in a target or a target sub-region of the patient.

28. The method of claim 21, wherein the initial optimization goals include a goal to decrease LET in an organ at risk or a sub-region of an organ at risk.

29. At least one machine-readable medium including instructions for radiotherapy treatment planning, which when executed by processing circuitry, cause the processing circuitry to perform operations to:
receive patient information corresponding to a patient;
determine a set of optimization functions with initial optimization goals, including at least one optimization function depending on linear energy transfer (LET) and at least one optimization function for selecting a dose;
generate a treatment plan via automated multicriteria optimization of the set of optimization functions while preserving the initial optimization goals using the patient information; and
output the treatment plan.

30. The at least one machine-readable medium of claim 29, wherein the treatment plan includes using proton arc therapy.

31. The at least one machine-readable medium of claim 29, wherein the treatment plan includes using intensity modulated proton therapy (IMPT).

32. The at least one machine-readable medium of claim 29, wherein to generate the treatment plan, the operations cause the processing circuitry to generate a robust optimization treatment plan, and wherein at least one beam of the robust optimization treatment plan is associated with multiple dose distributions, multiple LET distributions, or multiple LET surrogate distributions.

33. The at least one machine-readable medium of claim 29, wherein the at least one optimization function depending on LET includes using a LET surrogate.

34. The at least one machine-readable medium of claim 29, wherein the at least one optimization function depending on LET is a modified version of an existing dose cost functions.

35. The at least one machine-readable medium of claim 29, wherein the initial optimization goals include a goal to increase LET in a target or a target sub-region of the patient.

36. The at least one machine-readable medium of claim 29, wherein the initial optimization goals include a goal to decrease LET in an organ at risk or a sub-region of an organ at risk.

37. A system for radiotherapy treatment planning, the system comprising:
processing circuitry;
memory, including instructions, which when executed by the processing circuitry, cause the processing circuitry to:
receive patient information corresponding to a patient;
determine a set of optimization functions with initial optimization goals, including at least one optimization function depending on linear energy transfer (LET) and at least one optimization function for selecting a dose;
generate a treatment plan via automated multicriteria optimization of the set of optimization functions while preserving the initial optimization goals using the patient information; and
output the treatment plan.

38. The system of claim 37, wherein the treatment plan includes using proton arc therapy.

39. The system of claim 37, wherein the treatment plan includes using intensity modulated proton therapy (IMPT).

40. The system of claim 37, wherein the processing circuitry is further caused to receive a selection to optimize LET during treatment planning, and wherein to determine the set of optimization functions occurs in response to receiving the selection.
